(19) United States
(12) Patent Application Publication
(10) Pub. No.: US 2004/0166077 A1
(43) Pub. Date: Aug. 26, 2004

(54) Process for treating keratin materials with aromatic active principles

(75) Inventors: Leila Toumi, Sannois (FR); Anne-Laure Bernard, Courbevoie (FR); Henri Samain, Bievres (FR); Luc Nicolas Morgantini, Rully (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/724,828

(22) Filed: Dec. 2, 2003

Related U.S. Application Data
(60) Provisional application No. 60/459,641, filed on Apr. 3, 2003.

(30) Foreign Application Priority Data
Dec. 4, 2002 (FR) 0215298

(51) Int. Cl. A61K 7/06; A61K 7/11

(52) U.S. Cl. 424/70.11

(57) ABSTRACT

The present invention relates to a process for treating keratin materials, and especially human keratin materials such as the hair, the integuments and the skin, comprising the controlled release of an aromatic cosmetic active principle from synthetic polymer particles containing aryl groups, with a glass transition temperature (T_{g}) of greater than or equal to 45° C., and also to a kit for performing such a process.

### REFERENCE TO PRIOR APPLICATIONS

This application claims priority to U.S. provisional application 60/459,641 filed Apr. 3, 2003, and to French patent application 02 15298 filed Dec. 4, 2002, both incorporated herein by reference.

### FIELD OF THE INVENTION

The present invention relates to a process for treating keratin materials, and especially human keratin materials such as the hair, the integuments and the skin, comprising the controlled release of an aromatic cosmetic active principle from synthetic polymer particles containing aryl groups, with a glass transition temperature (T_{g}) of greater than or equal to 45° C., and also to a kit for performing such a process.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The use of cosmetic active agents that are sensitive to the formulation medium is a problem that has still not found a satisfactory solution. One approach generally consists in adding said active principle to the formulation medium at the time of use. However, this solution may have considerable drawbacks since it may require dissolution or homogenization steps that are difficult to perform in the context of a domestic use.

Moreover, this solution is unsatisfactory when the degradation of the active principle by one of the components of the formulation medium is so fast that a large fraction of the active principle is destroyed even before the treatment process. One concrete example of such a situation is the oxidation dyeing of the hair using both oxidation dye precursors (bases and couplers) and direct dyes. A process of oxidation dyeing of the hair conventionally involves the extemporaneous mixing of a dye solution containing oxidation dye precursors, which are colourless or weakly coloured, with an oxidizing agent that triggers an oxidative condensation of the precursors, resulting in the formation of coloured compounds of high mass at the surface of or in the keratin fibres.

Very frequently, and in particular for the production of shades with warm glints (coppery, violet or mahogany glints), “direct” dyes, which do not participate in the oxidation reaction but interact directly with the hair, are added to the oxidation dyes.

The oxidation dye precursors are stored in a separate compartment in the presence of a reducing agent, the function of which is to stabilize the composition by preventing the premature oxidation of the precursors. However, the most commonly used reducing agents, such as sodium metabisulphite and thiols, have the drawback of degrading a certain number of direct dyes, which prohibits the storage of the two types of dye (oxidation dyes and direct dyes) in the same compartment.

The Assignee has solved the problem of the instability of direct dyes in the presence of reducing agents, and more generally the problem posed by the use of cosmetic active principles that are sensitive to the formulation medium, by virtue of a cosmetic treatment process that involves the use of active principles enclosed in a particular protective matrix, followed by the gradual release of said active principle by chemical degradation of said protective matrix by means of a suitable reagent.

The incorporation of hydrophilic active principles, and in particular water-soluble active principles, into solid polymer particles is often problematic since the active principles are generally incompatible with the polymer matrix, and “sweating” is observed, i.e. a slow migration of the active principle molecules towards the external medium.

The encapsulation of active principles of low mass in standard microcapsules or nanocapsules—i.e. in particles consisting of a hydrophilic liquid phase surrounded by a polymer shell—obtained either by interface polymerization or by water/oil/water multiple emulsion followed by evaporation of the solvent, or by coacervation, is also found to be unsatisfactory in the majority of cases since the prepared capsules generally release the active principles more or less quickly into the external aqueous phase. Such a release is also observed for other types of organized structures, such as vesicles or multiple emulsions.

### DETAILED DESCRIPTION OF THE PREFERRED EMBODIMENTS

The Applicant has solved these problems for a particular group of active principles of low mass, i.e. for active principles comprising one or more aromatic nuclei, by incorporating these active principles into particles comprising a polymer that bears a number of aryl groups and that is in vitreous form at room temperature. This polymer, in order to be able to be degraded and to release the cosmetic active principle during the cosmetic treatment process, must comprise covalent bonds capable of being cleaved by a suitable reagent.

Consequently, one subject of the present invention is a cosmetic process for treating keratin materials, in particular human keratin materials such as the hair, the integuments and the skin, comprising the simultaneous or consecutive application of two compositions (a) and (b),

composition (a) containing particles comprising:

(i) at least one cosmetic active principle, which is preferably water-soluble, comprising one or more aromatic, carbocyclic or heterocyclic, monocyclic or fused polycyclic groups, with a molecular weight of less than or equal to 1000, and

(ii) at least one synthetic polymer containing aryl groups with a glass transition temperature (T_{g}) of greater than or equal to 45° C. and comprising covalent bonds capable of being cleaved by a reagent R present in composition (b), and

o composition (b) comprising at least one reagent R capable of cleaving certain covalent bonds of the aryl-containing polymer forming the particles of composition (a),

the chemical cleavage of the bonds of the aryl-containing polymer forming the particles present in composition (a) with the reagent R present in composition (b) resulting in a release of the aromatic cosmetic active principle.

The particles of the invention are either composite particles or capsules, also known as particles of core-shell structure. In the latter case, the wall or shell of the capsules consists of the polymer(s) containing aryl groups and the core contains the cosmetic active agent(s) in solid form. Preferably, the particles of the invention are composite particles, i.e. particles in which the cosmetic active agent(s) is (are) dispersed or dissolved in the polymer matrix.

A subject of the invention is also a kit for the cosmetic treatment of keratin materials, in particular human keratin materials, such as the hair, the integuments and the skin, comprising at least two compartments (A) and (B) that contain, respectively, compositions (a) and (b) as defined above.

As explained in the introduction, the process that is the subject of the present invention and the kit for implementing it are of particular advantage when the composition (a) also contains a chemical agent capable of degrading said aromatic cosmetic active principle. The presence of this chemical agent in composition (a) is, however, not essential for the invention since the agent capable of degrading the cosmetic active principle may just as easily be an external physicochemical agent such as ambient temperature or solar radiation. Thus, the process of the invention might serve to prolong the action of an antisun composition by gradually releasing photosensitive sunscreens that are photostabilized by encapsulation in the aryl-containing polymer particles described below.

The term “synthetic polymers containing aryl groups” used to denote the polymers used in the manufacture of the particles of the present invention encompasses polymers comprising a certain number of aryl and/or arylene groups, preferably phenyl and/or phenylene groups, the arylene groups possibly forming the main chain of the polymer or forming part of the side chains. They may be homopolymers or block, random or alternating copolymers, obtained by polyaddition or by polycondensation. This term also encompasses grafting polymers with a branched or crosslinked structure.

The aryl-containing polymers forming the matrix or shell of the particles of the present invention must, of course, be insoluble in water and in most cosmetically acceptable solvents. The aryl-containing polymers used in the present invention are consequently preferably nonionic, i.e. free of charges liable to make them soluble in water or in other polar solvents.

As indicated above, the glass transition temperature of the polymer forming the matrix or shell of the particles used in the process of the invention must be at least equal to 45° C. It is preferably greater than or equal to 50° C. and ideally greater than or equal to 60° C. This parameter is essential for the invention. Indeed, it has been found that the set, vitreous nature of the polymer at room temperature is an essential condition to prevent the migration of the encapsulated active principle molecules towards the surface of the particles, or the penetration of chemical agent into the particles. However, the particles used in the present invention are liable to be exposed to relatively high temperatures prevailing, for example, in summer on a beach. When the temperature of the particles then approaches the glass transition temperature of the polymer, the polymer becomes plastic and there is a risk of leakage of the hydrophilic active principle out of the particles.

As explained above, the aryl-containing polymer used to protect the aromatic active principles should not only allow satisfactory encapsulation of the active principle, but should also be degraded relatively quickly in the presence of a suitable reagent so as to allow the release of the active principle during the cosmetic treatment process.

In one preferred embodiment of the invention, the covalent bonds of the aryl-containing polymer that are liable to be cleaved by the reagent R are Si—C_{aromatic} bonds, such as Si-phenyl, and/or disulphide bonds (S—S bonds).

Indeed, the Applicant has observed that these two types of bond, even when they are part of the macromolecular chain of a solid polymer, which is insoluble in the reaction medium, may be rapidly cleaved, for example in the presence of hydrogen peroxide.

In the case of a process of oxidation dyeing of the hair—which is a preferred embodiment of the process of the invention—said hydrogen peroxide then acts both as the reagent R (degradation of the aryl-containing polymer) and as the oxidizing agent for the oxidative condensation of the oxidation dye precursors.

Another reagent R, capable of cleaving Si—C_{aromatic} bonds, includes fluoride ions, for example in the form of fluorine salts such as sodium, potassium or tetrabutyl-ammonium fluoride. The fluorine salts may also be used in combination with hydrogen peroxide (H₂O₂) and then increase the rate of cleavage of the Si—C_{aromatic} bonds and accelerate the release of the water-soluble active principle.

One group of preferred aryl-containing polymers used in the invention is formed by polyorganosiloxanes comprising aryl groups, preferably phenyl groups, which are borne directly by the silicon atoms of the siloxane skeleton. The phenyl-containing polyorganosiloxanes may have a linear, branched or crosslinked structure.

The appropriate content of aryl groups, and in particular phenyl groups, in the polyorganosiloxanes depends, inter alia, on the amount of active principle to be incorporated. Specifically, the inventors have found that for a given type of polymer, the amount of active principle that it is possible to encapsulate in a stable manner increases as the content of aryl groups increases. Moreover, the content of aryl groups has an influence on the glass transition temperature, which is generally higher for higher contents of aryl groups.

The inventors have obtained satisfactory results for an aryl group content such that the ratio of the number of aryl groups to the number of silicon atoms is between 1/15 and 2/1. Polyorganosiloxanes with a ratio of the number of aryl groups to the number of silicon atoms in the range from 1/10 to 2/1 are most particularly preferred.

Examples of such aryl-containing polyorganosiloxanes that may be mentioned include the polymer DC(R)Z-6018, which is a phenylpropylsilsesquioxane sold by the company Dow Corning.

Another family of aryl-containing polymers that may be used for the stabilization and controlled release of aromatic active principles includes polycondensates obtained from aryl-containing monomers comprising disulphide bonds. These polycondensates are generally copolymers consisting of at least two different types of monomer. The aryl groups and the S—S bonds may then be borne by the same monomer or by two or more than two different monomers.

Examples of monomers comprising an S—S bond or both an aryl group and an S—S bond include, for example, bis(4-aminophenyl) disulphide, homocystine, cystamine, form-amidine disulphide and bis(2-hydroxyethyl) disulphide.

Examples of such polycondensates that may be mentioned include the polyurethanes obtained by polycondensation of at least one diisocyanate and of at least one compound comprising two functions containing labile hydrogen, chosen, for example, from hydroxyl, thiol, primary amine and secondary amine functions, the monomers being chosen such that at least one type of comonomer comprises an aryl group and at least one comonomer comprises a disulphide bond (S—S). It emerges from the text hereinabove that the term “polyurethanes” as used in the present application encompasses not only polyurethanes per se comprising carbamate bonds (—NH—CO—O—) but also polyureas (containing —NH—CO—NH— or —NR—CO—NH— bonds) and polythioureas (containing —NH—CO—S— bonds).

The aryl-containing polycondensates may also be polyesters or polyamides obtained by polycondensation of at least one diacid or an activated derivative of a diacid, with, respectively, at least one diol or at least one diamine, these monomers being chosen, as previously, such that at least one of the types of monomer comprises an aryl group and at least one type of monomer comprises a disulphide bond.

The monomers giving the above-mentioned aryl-containing polycondensates, i.e. polyurethanes, polyesters, polyamides and copolymers thereof, are known in the art.

Examples of non-aryl-containing monomers that may be mentioned include:

aliphatic diisocyanates such as hexamethylene diisocyanate and isophorone diisocyanate,

aliphatic diols such as alkylene glycols or polyalkylene glycols,

aliphatic diamines such as alkylene diamines,

aliphatic diacids such as succinic acid, fumaric acid, adipic acid and sebacic acid, or the anhydrides or chlorides of these diacids,

amino alcohols,

amino acids.

Examples of aryl-containing monomers that may be mentioned include:

aromatic diisocyanates such as toluene diisocyanate and diphenylmethane diisocyanate,

aromatic diamines such as phenylenediamines, which are optionally substituted,

aromatic diols such as bisphenols, for example bisphenol A,

aromatic diacids such as terephthalic acid and isophthalic acid.

The polycondensation of the divalent monomers listed above results in linear structures. To obtain branched structures, a certain fraction of at least trivalent monomers may be added, such as 1,3,5-benzene-tricarboxylic acid trichloride, silicon tetraisocyanate, 2,4,6-triamino-1,3,5-triazine, glycerol, poly-glycerol or polypropoxylated glycerol containing amino end groups.

The particles of the present invention may have very variable structures and sizes, resulting from the process for preparing them and linked to the intended use.

They may be, for example, microparticles obtained by grinding a solid material consisting of a phenyl-containing polymer matrix in which the aromatic active principle(s) is (are) finely dispersed or dissolved (solid solution). These microparticles preferably have a mean size of between 0.05 and 500 μm.

Other microparticles with a size of between 0.05 and 500 μm may be obtained by multiple emulsion. They have a structure of the core-shell type in which a shell formed by the aryl-containing polymer hermetically surrounds a liquid core containing the active principle. The multiple emulsion technique used for the formation of particles of this type is described, for example, in FR 2 766 737 and in DE 197 19 297.

In another embodiment, the particles are particles with a core-shell structure that are relatively larger in size, preferably between 0.5 and 10 mm. These particles are obtained by coextrusion of two phases, the outer phase being formed by the aryl-containing polymer, in melt form or in the form of a viscous solution, preferably with a viscosity (measured at 20° C.) of greater than 1000 centipoises, and the inner phase containing the active principle in melt form or in the form of a thick solution.

To effectively protect the active principle in the polymer matrix of the particles of the invention, it is preferable for the weight ratio of the active principle to the aryl-containing polymer not to exceed a certain limiting value that depends, for example, on the content of aryl groups of the polymer. The higher this content, the higher the upper limit of the active principle/aryl-containing polymer weight ratio giving rise to effective protection. The Applicant has found that satisfactory encapsulation results are especially obtained for a weight ratio of the aromatic water-soluble active principle to the aryl-containing polymer of between 1/1 and 1/50 and preferably between 1/3 and 1/20.

The particles of aryl-containing polymer containing a water-soluble aromatic active principle may be prepared according to various known techniques comprising the incorporation of said aromatic active principle into a matrix of a synthetic polymer containing aryl groups or the coating or encapsulation of such an aromatic water-soluble active principle in a shell formed from such a polymer containing aryl groups.

The incorporation of the active principle into a matrix may be performed either by fusion or by dissolution of the polymer and the active principle.

The fusion process comprises the following steps consisting in:

melting the synthetic polymer containing aryl groups,

dissolving or finely dispersing the aromatic active principle in the molten polymer,

allowing the mixture thus obtained to cool to room temperature, and

grinding the solid material to a suitable particle size, preferably of between 0.05 and 500 μm.

This process has the advantage of being able to be performed in the absence of organic solvents. However, it has the difficulty of being able to be performed only with polymers whose melting point is less than the decomposition temperature of the active principle.

The dissolution process makes it possible to overcome this drawback. This process comprises the following steps:

dissolving the synthetic polymer containing aryl groups and the aromatic active principle in a suitable solvent or mixture of solvents,

evaporating off the solvent so as to obtain a solid material, and

grinding the solid material to a suitable particle size, preferably of between 0.05 and 500 μm.

This process requires the selection of a solvent or mixture of solvents that allows both the hydrophobic aryl polymer and the active principle, which is often hydrophilic, to be dissolved.

Examples of such solvents or mixtures of solvents that may be mentioned include C_{1-4 alcohols such as methanol, ethanol and isopropanol. Other organic solvents such as DMSO and tetrahydrofurane may also be used.}

The grinding of the vitreous solid material obtained after cooling to room temperature or after evaporating off the solvent may be performed using any type of mill known in the art that makes it possible to obtain particles of the desired size, preferably between 0.05 and 500 μm. Examples that may be mentioned include knife mills, ball mills and pin mills.

Finally, a third process comprises coextrusion of the aromatic water-soluble active principle and of the synthetic polymer containing aryl groups so as to obtain particles consisting of a solid core formed by the aromatic water-soluble active principle, and of a solid shell formed by the synthetic polymer containing aryl groups.

The coextrusion of the aryl-containing polymer and of the aromatic water-soluble active principle is performed according to a known process described, for example, in Harper J. M., 1990, Extrusion of foods in biotechnology and food process engineering, published by Marcel Dekker Inc., Chapter 10, page 307.

The cosmetic active principles are chosen from aromatic active principles of low molecular weight, i.e. with a molecular weight of less than 1000 and preferably less than 500. These aromatic active principles of low mass are preferably water-soluble.

According to the invention, the expression “water-soluble active principle” means an active principle with a solubility in water, measured at 25° C., at least equal to 0.1 g/l (production of a macroscopically isotropic and transparent, coloured or uncoloured solution). This solubility is preferably greater than or equal to 1 g/l.

The hydrophilic nature of the aromatic active principles may be provided by one or more ionizable or ionized substituents, such as sulphonate, sulphate, carboxylate, phosphonate, phosphate, primary, secondary or tertiary amine groups or an ammonium group.

Examples of such cosmetic active principles that may be mentioned include:

dyes such as direct dyes and oxidation dye precursors (bases or couplers),

organic UV-A and UV-B screening agents, such as dibenzoylmethanes, benzimidazoles, benzophenones and benzotriazoles,

flavonoids such as those contained in rye extracts,

vitamins such as vitamin B12,

reducing agents,

acids such as salicylic acid.

Among these active principles, the ones that are particularly preferred are direct hair dyes and in particular azo direct dyes, nitro direct dyes, anthraquinone or naphthalene direct dyes, triarylmethine direct dyes, organic UV-screening agents and flavonoids. The direct dyes may be of nonionic, anionic, cationic or amphoteric nature.

As explained in the introduction, the cosmetic treatment process of the present invention is particularly advantageous in the field of oxidation dyeing of the hair using both oxidation dye precursors and direct dyes that are sensitive to the reducing agent (for example sodium metabisulphite) that is intended to prevent premature oxidation of the oxidation dye precursors.

Consequently, in one particularly preferred embodiment of the process of the invention, the following are mixed together, immediately before use:

a dye composition comprising, in a cosmetically acceptable medium, one or more oxidation dye precursors, a reducing agent and an aromatic dye, which is preferably water-soluble and which can be degraded by said reducing agent, this dye being enclosed in particles of a synthetic polymer containing aryl groups, with a glass transition temperature (T_{g}) of greater than or equal to 45° C. and comprising S—S bonds and/or Si—C_{aromatic} bonds, and

an oxidizing composition containing hydrogen peroxide,

the mixture thus obtained is applied to keratin fibres, it is left in place for a time that is sufficient to obtain the desired coloration, and the keratin fibres are rinsed, washed and dried.

A subject of the invention is also multi-compartment kits for performing the cosmetic treatment process described above. Such a multi-compartment kit according to the invention comprises at least two compartments (A) and (B), compartment (A) containing a composition (a) including particles comprising:

(i) at least one cosmetic active principle, which is preferably water-soluble, comprising one or more aromatic, carbocyclic or heterocyclic, monocyclic or fused polycyclic groups, with a molecular weight of less than or equal to 1000, and

(ii) at least one synthetic polymer containing aryl groups, with a glass transition temperature (T_{g}) of greater than or equal to 45° C. and comprising covalent bonds that may be cleaved with a reagent R present in composition (b),

and compartment (B) containing a composition (b) that contains at least one reagent R capable of cleaving certain covalent bonds of the aryl-containing polymer forming the particles of composition (a).

In one preferred embodiment of the invention, the multi-compartment kit is a kit for the oxidation dyeing of the hair, in which composition (a) contains, in a cosmetically acceptable medium, one or more oxidation dye precursors, a reducing agent and an aromatic dye, which is preferably water-soluble and which can be degraded by the reducing agent, this dye being enclosed in particles of a synthetic polymer containing aryl groups, with a glass transition temperature (T_{g}) of greater than or equal to 45° C. and comprising S—S bonds and/or Si-phenyl bonds, and composition (b) contains hydrogen peroxide.

The examples below illustrate the preparation of particles of aryl-containing polymer containing a water-soluble aromatic active principle (Example 1) and a cosmetic treatment process with controlled release of the active principle by chemical degradation of the particles containing this active principle (Example 2).

### EXAMPLE 1

Encapsulation of a Water-Soluble Cosmetic Active Principle

1 part by weight of a water-soluble azo dye powder with a molecular mass of less than 300 g/mol (MIP Red 2985-2, Ciba) is dispersed in 10 parts by weight of a phenylpropylsilsesquioxane with a mass of 1500-2500 g/mol (DC® Z-6018, Dow Corning) in molten form (130° C.) and the mixture is mixed until a homogeneous suspension is obtained. The mixture is allowed to cool to room temperature and is then ground using a mortar, to a mean particle size of about 500 μm. The powder thus prepared is then washed 3 times, for 24 hours with stirring, in an aqueous solution, and is then dispersed in water using a silicone dispersant (dimethicone copolyol isostearate).

The object of this washing is to remove the dye molecules that are at the surface of the ground particles. An assay of the amount of dye contained in the particles thus washed reveals that 8% of the dye has been removed by washing, i.e. the degree of encapsulation is 92%.

Efficacy of Protection of the Encapsulated Dye

The powder is then dispersed, using the above silicone dispersant, in an ammoniacal solution at pH 10.5, containing 0.53% by weight of sodium metabisulphite, and in an ammoniacal solution at pH 10.5 free of reducing agent.

After storing these two suspensions at a temperature of 45° C. for 2 months, the assay of the amount of dye that has leaked into the metabisulphite-free ammoniacal aqueous medium indicates a degree of encapsulation of 90% instead of the 92% initially measured, i.e. a loss of about 2% of the amount of dye initially encapsulated.

The assay of the amount of dye remaining in the particles stored for 2 months at 45° C. in a sodium meta-bisulphite solution, by UV-visible spectrometry of a solution of the powder in a chloroform/methanol mixture (9/1), reveals a loss of dye of about 5% (87% of dye remaining instead of the 92% initially encapsulated).

The fact that the loss of dye in the presence of reducing agent (5%) is slightly higher than that observed in the absence of reducing agent (2%) shows that a small fraction of encapsulated dye has been destroyed by the reducing agent. This result may be attributed to the very large specific surface area of the finely dispersed powder, which promotes exchanges between the surface of the particles and the surrounding medium.

For comparative purposes, if the dye is introduced directly into the ammoniacal solution at pH 10.5 containing the sodium bisulphite (thus without protection by the particles), it is found that it is totally destroyed after 2 months at 45° C.

This example shows that the dye is encapsulated with a good degree of encapsulation and is effectively protected against chemical degradation by the sodium metabisulphite.

### EXAMPLE 2

Hair Dyeing Kit and Dyeing Process

| Compartment A | | |
|---|---|---|
| Direct dye (MIP Red 2985-2, Ciba) encapsulated | 0.1 | g a.m. |
| in accordance with Example 1 | | |
| NH₄OH | 2 g | |
| Sodium metabisulphite | 0.53 | g a.m. |
| Water | qs 100 | g |
| Compartment B | | |
| Aqueous hydrogen peroxide solution | 20 | volumes |

1 part by volume of the composition of compartment A is mixed with 1 part by volume of the composition of compartment B. The mixture obtained has a pH of 9.53. After 10 minutes, the mixture is applied to a lock of permanent-waved hair containing 90% white hairs, with a dye composition/lock weight ratio of about 10.

After a lead-in time of 30 minutes at room temperature, the lock is rinsed with water and dried.

Table 1 below collates the calorimetric parameters of the CIE L*a*b* system and of the HSL (Hue, Saturation, Luminosity) system, measured using a Minolta CM3600d calorimeter (illuminant D65-10° CSE).

The difference in colour ΔE between the original lock and the dyed lock is calculated according to the formula

ΔE=\sqrt{(L*_{final}−L*ᵢₙᵢₜᵢₐₗ)²+(a*_{final}−a*ᵢₙᵢₜᵢₐₗ)²+(b*_{final}−b*ᵢₙᵢₜᵢₐₗ)²}

The values obtained with the dye composition according to the invention, prepared in the manner described above, are compared with those obtained, under the same conditions, with a mixture of the composition of compartment A above and of a solution of water acidified with phosphoric acid, free of reagent R (H₂O₂) capable of degrading the polymer forming the particles. The pH of the comparative mixture applied to the hair is 9.85.

| | L* | a* | b* | h (hue) | ΔE |
|---|---|---|---|---|---|
| Before dyeing | 59.97 | 1.82 | 11.12 | 80.70 | |
| Dyeing according to | 57.43 | 5.78 | 8.52 | 55.86 | 5.37 |
| the invention | | | | | |
| Comparative dyeing | 59.56 | 1.83 | 9.83 | 79.45 | 1.35 |

This example clearly shows that the particles placed in contact with the hydrogen peroxide degrade quickly and release the encapsulated direct dye.

In order to demonstrate the release of the direct dye encapsulated in an aryl-containing polymer, this example was performed with a direct dye in the absence of oxidation dye precursors.

It goes without saying that the release of the direct dye by the particles of aryl-containing polymer in contact with hydrogen peroxide may advantageously be exploited in an oxidation dyeing process using known oxidation dye precursors (bases and couplers), which would then be present in compartment A of the kit used above.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for treating keratin material, comprising the simultaneous or consecutive application to keratin material of two compositions (a) and (b):
composition (a) comprising particles comprising:
(i) at least one cosmetic active principle comprising one or more aromatic, carbocyclic or heterocyclic, monocyclic or fused polycyclic groups, with a molecular weight of less than or equal to 1000, and
(ii) at least one synthetic polymer containing aryl groups with a glass transition temperature (T_{g}) of greater than or equal to 45° C. and comprising covalent bonds capable of being cleaved by a reagent R present in composition (b),
composition (b) comprising at least one reagent R capable of cleaving at least one covalent bond of the synthetic polymer containing aryl groups in the particles of composition (a),
the chemical cleavage of at least one covalent bond of the synthetic polymer containing aryl groups in the particles of composition (a) by reagent R resulting in a release of the aromatic cosmetic active principle.

2. The process according to claim 1, wherein the cosmetic active principle(s) (i) is (are) water-soluble.

3. The process according to claim 1, wherein the particles are composite particles in which the cosmetic active principle(s) is (are) dispersed or dissolved in the polymer matrix.

4. The process according to claim 1, wherein composition (a) further comprises a chemical agent capable of degrading said cosmetic active principle.

5. The process according to claim 1, wherein the synthetic polymer containing aryl groups is a nonionic polymer.

6. The process according to claim 1, wherein the glass transition temperature of the synthetic polymer containing aryl groups is greater than 50° C.

7. The process according to claim 1, wherein the covalent bonds of the aryl-containing polymer which are capable of being cleaved by the reagent R are Si—C_{aromatic} bonds and/or disulphide bonds (S—S).

8. The process according to claim 7, wherein the polymer containing aryl groups is a polyorganosiloxane comprising aryl groups linked directly to silicon atoms of a siloxane skeleton.

9. The process according to claim 8, wherein the ratio of the number of aryl groups to the number of silicon atoms in the polymer is 1/15-2/1.

10. The process according to claim 7, wherein the synthetic polymer containing aryl groups is a polyurethane obtained by polycondensation of at least one diisocyanate and of at least one compound comprising two functions containing labile hydrogen, these monomers being chosen such that at least one type of comonomer comprises an aryl group and at least one type of comonomer comprises a disulphide bond (S—S).

11. The process according to claim 7, wherein the synthetic polymer containing aryl groups is a polyester or polyamide obtained by polycondensation of at least one diacid or of an activated derivative of a diacid, and, respectively, of at least one diol or of at least one diamine, these comonomers being chosen such that at least one type of comonomer comprises an aryl group and at least one type of comonomer comprises a disulphide bond (S—S).

12. The process according to claim 1, wherein the reagent R is hydrogen peroxide (H₂O₂).

13. The process according to claim 1, wherein the aromatic cosmetic active principle has a molecular weight of less than or equal to 500.

14. The process according to claim 1, wherein the aromatic cosmetic active principle bears at least one ionized or ionizable group.

15. The process according to claim 1, wherein the weight ratio of the cosmetic active principle to the aryl-containing polymer is 1/1-1/50.

16. The process according to claim 1, wherein the cosmetic active principle is a direct hair dye, an oxidation dye precursor, an organic UV-screening agent or a flavonoid.

17. The process according to claim 1, wherein the particles present in composition (a) are microparticles with a mean size of 0.05-500 μm.

18. The process according to claim 17, wherein the microparticles have a core-shell structure in which the core is formed from or contains said aromatic cosmetic active principle and the shell comprises or is formed from said synthetic polymer containing aryl groups.

19. The process according to claim 1, wherein the following are mixed together, immediately before use:
a dye composition comprising, in a cosmetically acceptable medium, one or more oxidation dye precursors, a reducing agent and an aromatic dye which can be degraded by said reducing agent, this dye being enclosed in particles of a synthetic polymer containing aryl groups, with a glass transition temperature (T_{g}) of greater than or equal to 45° C. and comprising S—S bonds and/or Si—C_{aromatic} bonds, and
an oxidizing composition comprising hydrogen peroxide,
and wherein the mixture thus obtained is applied to keratin fibres, is left in place for a time that is sufficient to obtain the desired coloration, and the keratin fibres are then rinsed, washed and dried.

20. The process according to claim 19, wherein the reducing agent is sodium metabisulphite or a thiol.

21. The process according to claim 1, wherein the glass transition temperature of the synthetic polymer containing aryl groups is greater than 60° C.

22. The process according to claim 1, wherein the weight ratio of the cosmetic active principle to the aryl-containing polymer is 1/3-1/20.

23. The process according to claim 1, wherein the particles present in composition (a) are particles with a mean size of 0.5-10 mm.

24. A kit for the cosmetic treatment of keratin material, comprising at least two compartments (A) and (B) wherein present in said compartments, respectively, are compositions (a) and (b) as defined in claim 1.

25. The kit according to claim 21, wherein composition (a) comprises, in a cosmetically acceptable medium, one or more oxidation dye precursors, a reducing agent and an aromatic dye, this dye being enclosed in particles of a synthetic polymer containing aryl groups, with a glass transition temperature (T_{g}) of greater than or equal to 45° C. and comprising S—S bonds and/or Si-phenyl bonds, and composition (b) comprises hydrogen peroxide.
